(19) United States
(12) Patent Application Publication
(10) Pub. No.: US 2026/0232483 A1
(43) Pub. Date: Aug. 13, 2026

(54) HEAT TRANSFER UNIT AND ASSOCIATED APPARATUS

(71) Applicant: HILOTHERM HOLDING AG, Oberwil b. Zug (CH)

(72) Inventor: Christian STEGMANN, Burgberg im Allgäu (DE)

(21) Appl. No.: 19/157,930

(22) PCT Filed: Feb. 19, 2024
(86) PCT No.: PCT/EP2024/054115

§ 371 Date: Aug. 19, 2025

(30) Foreign Application Priority Data
Feb. 21, 2023 (LU) 503521

(51) Int. Cl. A61F 7/00; A61F 7/02

(52) U.S. Cl. CPC A61F 7/0053; A61F 7/0085; A61F 7/02; A61F 2007/0045; A61F 2007/0056; A61F 2007/0231; A61F 2007/0274

(57) ABSTRACT

A heat transfer unit is adapted to be applied to a human foot in order to transfer heat to/from the human foot. The heat transfer unit comprises a first flexible material layer and a second flexible material layer defining a fluid chamber therebetween, allowing a heat transfer fluid to flow through the fluid chamber. The first and second flexible material layers are connected to one another at a plurality of connection locations. In a first region of the heat transfer unit, the connection locations have a first average spacing and, in a second region of the heat transfer unit, the connection locations have a second average spacing, wherein the first average spacing is smaller than the second average spacing. In normal use of the heat transfer unit, the first region of the heat transfer unit is arranged to be located adjacent a sole of the human foot, and the second region of the heat transfer unit is arranged to be located adjacent a region of the human foot different from the sole of the human foot.

This application claims priority to Luxemburg Patent Application No. LU 503521 filed Feb. 21, 2023, the entire contents of which are hereby incorporated by reference.

The present invention relates to a heat transfer unit and a heat transfer apparatus for use in the thermal therapy treatment of a human foot, and in particular for use in the treatment of tissue damage after surgical interventions, of injuries, of inflammations and of chronic diseases of the rheumatic type, and in particular for assisting chemotherapeutic treatments, as well as a method of heating or cooling a human foot.

In the context of the present invention, the term “thermal therapy treatment” is intended to be understood to mean a treatment in which heat is supplied to the human foot, or removed therefrom.

Preferably, this is not a matter of supplying heat to, or removing heat from, the entire body, as is done, for example, in a sauna and the subsequent immersion in a plunge pool filled with cold water, but it is preferably a matter of supplying heat specifically to, or removing heat specifically from, the foot, in order to achieve a therapeutic effect, preferably without significantly supplying heat to, or removing heat from, the remainder of the human body.

WO2022/101320 discloses a heat transfer unit and a heat transfer apparatus of the kind mentioned above.

It is an object of the present invention to provide an alternative heat transfer unit and heat transfer apparatus, in particular a further improved heat transfer unit and heat transfer apparatus, as well as an associated method.

Aspects of the present invention are set out in the claims. Each feature disclosed and/or illustrated in the present specification may be incorporated in the invention, whether alone or in combination with any other feature disclosed or illustrated herein, unless such combination is explicitly excluded or technically impossible. This relates to apparatus aspects and method aspects alike, as well as their preferred embodiments. Accordingly, for example, any of the devices disclosed herein can be used in the context of any of the methods disclosed herein, and vice versa.

According to a first aspect, the present invention provides a heat transfer unit adapted to be applied to a human foot in order to transfer heat to/from the human foot, wherein the heat transfer unit comprises a first flexible material layer and a second flexible material layer defining a fluid chamber therebetween, allowing a heat transfer fluid to flow through the fluid chamber,
wherein the first and second flexible material layers are connected to one another at a plurality of connection locations,
wherein, in a first region of the heat transfer unit, the connection locations have a first average spacing and, in a second region of the heat transfer unit, the connection locations have a second average spacing, wherein the first average spacing is smaller than the second average spacing,
wherein, in normal use of the heat transfer unit, the first region of the heat transfer unit is arranged to be located adjacent a sole of the human foot, and the second region of the heat transfer unit is arranged to be located adjacent a region of the human foot different from the sole of the human foot.

The first and second flexible material layers may be substantially fluid tight so as to form a substantially fluid tight fluid chamber (apart from any external connections described below). The first and second flexible material layers may be made from the same, or different materials, for example plastics material such as low-density polyethylene (LDPE). Other examples include polyurethane. The first and second flexible material layers may, for example, have a thickness of at least 0.1, 0.15, 0.2 or 0.25 mm and/or at most 0.5, 0.45, 0.4, 0.35 or 0.3 mm.

Since the first and second materials are flexible, the heat transfer unit can be at least partially wrapped around the foot to result in at least partial contact of the heat transfer unit, in particular the first flexible material layer, with the foot. At least some embodiments can enable better, or more complete, contact between the heat transfer unit and the foot than has been possible with prior art heat transfer units.

When a heat transfer fluid that is warmer than the foot is caused to flow through the fluid chamber, heat is transferred to the foot, thereby heating the foot. When a heat transfer fluid that is colder than the foot is caused to flow through the fluid chamber, heat is transferred from the foot, thereby cooling the foot.

The connection locations at which the first and second material layers are connected to one another are individual, relatively small, regions and may, for example, be substantially round or of any other suitable shape. The connection locations are sometimes also referred to as “dots”.

In order to connect the first and second material layers at the connection locations, the first and second material layers may be fused or welded together at selected locations. Alternatively, adhesive material may be used to connect the first and second material layers at the connection locations. Suitable methods of connecting the first and second material layers are, in principle, known to those skilled in the art and will not be described in detail.

In addition to being connected at the (individual) connection locations, the first and second material layers are connected at their periphery, in particular all along their periphery, with the possible exception of any external fluid connections-although any external fluid connections may alternatively be provided only in the first and/or second material layer, in which case the first and second material layers may be connected all along their periphery without interruption. The first and second material layers may be connected at their periphery using the same or a similar technique as the technique used for connecting the first and second material layers at the connection locations. In particular, the joining of the first and second material layers at the connection locations and along their periphery may be performed simultaneously, in particular using the same processing apparatus.

The connection locations serve to locate the first and second material layers with respect to one another. Individual spaces or passageways for the heat transfer fluid are formed between the connection locations, as well as between the connection locations and the periphery of the heat transfer unit. Accordingly, the connection locations should be kept relatively small, so as not to obstruct unduly the flow of the heat transfer fluid. On the other hand, the connection locations may need to be of sufficient size to ensure a firm connection between the first and second material layers.

The average spacing between the connection locations in the first and second regions can be determined in a variety of ways. For example, a square or round area of the heat transfer unit (or of the first and second flexible material layers) can be considered, whereby this area should have at least 4, 5, 6 or more connection locations. For each connection location, the nearest neighbor or neighbors (nearest neighboring connection location or locations) should be determined, as well as the distance between the respective connection location and its nearest neighbor(s). The average of these distances then indicates the average spacing between the connection locations in that region. Alternatively, the average density of connection locations within a particular area could be determined, for example by determining the number of connection locations in the respective area of the heat transfer unit, or the total surface area occupied by the connection locations in the respective area of the heat transfer unit. The number of connection locations in the respective area of the heat transfer unit or the total surface area occupied by the connection locations in the respective area of the heat transfer unit can then be put in relation to the surface area under consideration so as to provide a measure of the average spacing between the connection locations in the respective area of the heat transfer unit. Other ways of determining the average spacing between the connection locations may be conceivable. The inventor has found that, by providing first and second regions with a different average spacing between the connection locations, the heat transfer unit can be used more efficiently or effectively when compared with some prior art approaches. In particular, the inventor has firstly realized that a relatively dense pattern of the connection locations (i.e. relatively small average spacing between connection locations) occupies more surface area of the heat transfer unit than a less dense pattern of connection locations (i.e. relatively large average spacing between connection locations). A relatively dense pattern of connection locations therefore leaves less space for flow passages between the connection locations so that this reduces the width of these flow passages, thereby also reducing the volume of the fluid chamber in the first region. Secondly, he has realized that the relatively dense pattern of connection locations also reduces the extent to which the first and/or second material layers can “bulge out” between the connection locations, thereby also reducing the volume of the fluid chamber in the first region. He has further realized that the reduction of the volume of the fluid chamber in one region compared with another region can be used to a particular advantage: The sole of the foot is relatively flat, and therefore, when in use, the heat transfer unit is not subjected to relatively sharp bends in the region of the sole of the foot. By contrast, in other regions, the heat transfer unit may be subjected to relatively sharp bends when the heat transfer unit is wrapped relatively tightly around the foot to ensure good contact between the heat transfer unit and the foot. Increasing the cross-section of the flow passages in areas other than the sole of the foot can ensure that the heat transfer unit can adapt relatively well to the shape of the foot, thereby resulting in good contact between the heat transfer unit and the foot. An increased cross-section of the flow passages in the second region (and, as a result, an increase in the volume of the fluid chamber in the second region) is therefore beneficial in that it may help to ensure good contact between the heat transfer unit and the foot. As the heat transfer unit is less likely to be subjected to relatively sharp bends in the region of the sole of the foot, relatively good contact between the heat transfer unit and the foot can be achieved in the region of the sole of the foot despite a reduced cross section of the flow passages in the first region. In other words, in the region of the sole of the foot, there is no particular need for the cross-section of the flow passages to be made relatively large, and therefore the volume of the fluid chamber can be decreased in the region of the sole of the foot whilst maintaining good contact between the heat transfer unit and the foot. The decrease in volume of the fluid chamber in the region of the sole of the foot also means that the total volume of the fluid chamber (i.e. when the first and second regions are considered together) can be made smaller than if the heat transfer unit had a uniform average spacing of connection locations throughout, i.e. if the relatively large spacing of connection locations in the second region was also applied to the first region. This also means that the amount of heat transfer fluid required to (substantially) fill the fluid chamber can be reduced.

In turn, the reduction of the amount of heat transfer fluid required has another beneficial effect. Typically, when a heat transfer unit is used to heat or cool a foot, the heat transfer unit is initially applied to the foot while the heat transfer unit is substantially empty (i.e. little or no heat transfer fluid present in the heat transfer unit), which means that the required amount of heat transfer fluid initially needs to be stored in a heating or cooling device which is intended to supply the heat transfer fluid to the heat transfer unit (via suitable hoses). Subsequently, the heat transfer fluid is supplied to the heat transfer unit until the heat transfer unit is substantially filled with the heat transfer fluid, thereby decreasing the volume of heat transfer fluid in the heating or cooling device. Only when the heat transfer unit is substantially filled would any heat transfer fluid circulate from the heat transfer unit back to the heating or cooling device. To ensure that the heating or cooling device does not “run dry”, the heating or cooling device would either have to be dimensioned sufficiently large to hold at least the required amount of heat transfer fluid to fill the fluid chamber of the heat transfer unit, or it might be necessary to replenish an amount of heat transfer fluid into the heating or cooling device during operation. Reducing the total volume of the fluid chamber of the heat transfer unit may therefore mean that the volume of the heating or cooling device can be reduced accordingly, without there being a need to add an amount of heat transfer fluid during operation.

The second region (of the heat transfer unit) may be made up of several non-contiguous sub-regions. The same applies to the first region (of the heat transfer unit). For example, the second region may comprise a region attached to the left side of the first region and a separate region attached to the right side of the first region. Together, they form the second region.

In an ideal scenario, the first region, in which the connection locations have a first average spacing, comes to be located adjacent a sole of the human foot only, i.e. not (also) adjacent other surfaces of the foot (such as the sides or the dorsum of the foot, i.e. the top of the foot). Similarly, in this ideal scenario, the second region, in which the connection locations have a second average spacing, comes to be located only adjacent to those surfaces of the human foot which are different from the sole of the foot (such as the sides or the dorsum of the foot). However, given that human feet vary in terms of size and/or shape, this ideal scenario will rarely exist in reality. Nevertheless, at least some advantages of the present invention can be achieved even if a certain percentage (e.g. up to 10% or 20%) of the first region comes to be located adjacent a surface of the foot other than the sole of the foot, and if a certain percentage (e.g. up to 10% or 20%) of the second region comes to be located adjacent the sole of the foot.

In one embodiment, in normal use, the heat transfer unit is arranged to be bent along one or more bending lines or bending regions resulting in the heat transfer unit having radii of curvature respectively associated with each bending line or bending region, wherein the one or more bending lines or bending regions having the smallest radius of curvature are located in the second region, in particular wherein the radius of curvature is measured at a surface of the heat transfer unit facing away from the human foot, or in the middle between the first and second flexible material layers,
in particular wherein, in normal use:
the one or more bending lines or bending regions having the smallest radius of curvature are located along the front half of the left and/or right side of the human foot and/or along the Achilles tendon; and/or
the smallest radius of curvature is 2 cm or less, in particular 1.5 cm or less, in particular 1.3 cm or less.

The second region may, for example, comprise, or consist of, a region along the front half of the left and/or right side of the human foot, in particular a region along the front half of the left and/or right side of the human foot but excluding the toe region. The latter does not mean that, in embodiments where the second region comprises a region along the front half of the left and/or right side of the human foot but excluding the toe region, the toe region must not be part of the second region (where the average spacing of the connection locations is greater). It does however mean that a region along the front half of the left and/or right side of the human foot but which is different from the toe region must (also) form part of the second region.

The bending lines or bending regions are not necessarily marked on the heat transfer unit, and the heat transfer unit is not (necessarily) provided with features that cause the heat transfer unit to be bent along any particular line when it is being wrapped around a human foot. Instead, the bending lines or bending regions are lines or regions along which the heat transfer unit will be bent when it is being wrapped around a human foot. Their position and/or orientation therefore depends on the size and/or shape of the foot. To this end, the second region is sufficiently large and positioned such that the heat transfer unit can be applied to a variety of feet having different sizes and/or shapes whilst it is ensured that, over a given range of sizes and/or shapes, the bending lines or bending regions will be in the second region.

The absence of features that would cause the heat transfer unit to be bent along a particular line helps to prevent the heat transfer unit from collapsing along that line, i.e. it helps to prevent the first and second material layers from touching each other along that line, or from being in such close proximity to one another that the flow of heat transfer fluid across that line is significantly impeded.

The heat transfer unit has a greater spacing or lower density of connection locations in a region (i.e. the second region) where a heat transfer unit according to the prior art would be more likely to kink and restrict the fluid flow when it is being wrapped around a human foot, in particular along the front half of the sides of the foot and along the Achilles tendon. Whilst the region at the tip of the toes of an average foot may result in a similarly sharp “edge” so that kinking could normally be expected in that region as well, certain embodiments of the present invention envisage that portions on the left and right side of the heat transfer unit are first folded over so as to come to lie on top of the dorsum of the foot, and thereafter a further portion of the heat transfer unit at the front of the heat transfer unit is folded back so as to come to lie on top of the folded-over side portions. This means that the “edge” at the tip of the toes becomes less sharp, and therefore there is a reduced need for a larger spacing of the connection locations in the region of the heat transfer unit that would normally be folded over the toes. In other embodiments, the spacing of the connection locations in the region of the heat transfer unit that would normally be folded over the toes can also be increased when compared with the spacing of the connection locations in the first region.

For the purpose of the present specification, the expression “folding” (of the heat transfer unit) is not to be understood in the sense of folding a thin piece of paper, which would result in a very sharp edge. Instead, the expression “folding” is to be understood in the sense of bending the heat transfer unit (including over the dorsum of a foot) to result in radii of curvature significantly larger than in the case of folding a thin sheet of paper.

In one embodiment, at the one or more bending lines or bending regions having the smallest radius of curvature, connection locations that are nearest neighbors do not, in normal use of the heat transfer unit, form a line which is aligned with the respective bending line or with a longitudinal axis of the respective bending region, in particular wherein a line formed by nearest neighbors of connection locations includes an angle of at least 5°, 10°, 15°, 20°, 25° or 30° with said respective bending line or said longitudinal axis. This may further help to prevent the heat transfer unit from collapsing along a bending line or bending region.

In normal use, the heat transfer unit may be arranged to be bent along one or more bending lines or bending regions resulting in the heat transfer unit having radii of curvature respectively associated with each bending line or bending region, wherein the one or more bending lines or bending regions having the smallest radius of curvature are located in the second region, wherein, at the one or more bending lines or bending regions having the smallest radius of curvature, there are no straight lines
which are fitted through connection locations that are nearest neighbors and
which, in normal use of the heat transfer unit, are aligned with the respective bending line or with a longitudinal axis of the respective bending region, in particular wherein all such straight lines include an angle of at least 5°, 10°, 15°, 20°, 25° or 30° with said respective bending line or said longitudinal axis.”

According to some embodiments, in order to determine a line formed by nearest neighbors of connection locations, it is possible, for example, to consider a series of at least three, four, five or more nearest neighbors. According to such embodiments, a “group” of only two nearest neighbors of connection locations does not constitute such a series and is therefore not considered to form a line that, aligned with a respective bending line or longitudinal axis of a respective bending region, would be likely to result in the heat transfer unit collapsing. Ideally, however, none of the connection locations that are nearest neighbors form a line which is aligned with a respective bending line or with a longitudinal axis of a respective bending region.

In one embodiment, the first region occupies an area that represents a fraction of the total area of the heat transfer unit, wherein said fraction is between an upper limit and a lower limit, wherein the upper limit is 55%, 50% or 45%, and wherein the lower limit is 25%, 30% or 35%, in particular wherein said fraction is about 40%. The (majority of the) remainder of the heat transfer unit is occupied by the second region, e.g. between 75% and 45%, for example about 60%.

In one embodiment, the second region comprises one or more, in particular all of:
a rear left portion;
a front left portion;
a front right portion; and
a rear right portion.

As used herein, the terms “front”, “rear”, “left”, “right” and similar are intended to be understood in the context of a foot placed on a heat transfer unit in a substantially flat, level condition. The “rear” of the heat transfer unit would then be near the heel of the foot, the front of the heat transfer unit near the toes etc.

The front/rear left/right portions are not only connected to the first portion but are also in fluid communication with the first portion such that the heat transfer fluid can flow not only through the first portion but also through the front/rear left/right portions. However, the flow of heat transfer fluid does not necessarily need to occur over the entire length of the interface between the first portion and the front/rear left/right portions. Instead, barriers or compartmentalization lines (explained in more detail below) may be present at the interface between the first portion and the front/rear left/right portions. Further, a gap may exist between the first portion and part of the left/right portions when the heat transfer unit is in a flat condition, e.g. the front left portion may be (directly) connected to the first portion whilst the rear left portion is not (directly) connected to the first portion, even though an edge of the rear left portion may be located close to an edge of the first portion. Using again the rear left portion as an example, the rear left portion -or part thereof, in particular a rear part thereof-can be wrapped around the heel/Achilles tendon of the foot after the left portion (including the rear left portion) has been bent upwards such that the left portion covers (part of) the left side of the foot. The same applies to the (rear) right portion, mutatis mutandis.

In one embodiment, the heat transfer unit comprises one or more fasteners, in particular three or four fasteners, in particular hook-and-loop fasteners, in particular at a periphery of the heat transfer unit, for temporarily attaching a portion of the heat transfer unit to another portion of the heat transfer unit. One or more of the fasteners-whether they be of the hook-and-loop type or of another type-may or may not be stretchable. Use of stretchable fasteners my result in a tighter fit of the heat transfer unit with respect to the foot. For example, the fasteners may comprise a stretchable portion and a substantially non-stretchable portion, whereby the non-stretchable portion is advantageously located towards the “free” end of the fastener (e.g. the region which is intended to attach temporarily to another region of the heat transfer unit) and the stretchable portion is advantageously located between the non-stretchable portion of the fastener and the end of the fastener which is (permanently) fixed to the first and second flexible material layers.

In some embodiments, the heat transfer units may include five or more fasteners.

Depending on the size of the foot, the heat transfer unit, when applied to the foot (i.e. wrapped around the foot), may result in an overlap between portions of the heat transfer unit. Ideally, the transfer unit is dimensioned such that such an overlap results with all feet sizes for which the heat transfer unit is intended. This may help to ensure that as much of the surface of the foot is in close contact with the heat transfer unit.

Several sizes of heat transfer units may be provided in order to ensure that a heat transfer unit is available for a relatively large range of foot sizes/shapes, in particular not only for adults but also (young) children or even babies. However, given that heat transfer units according to embodiments of the present invention may have more flexibility and adjustability than heat transfer units according to the prior art, it may be possible to cover a given range of foot sizes/shapes with fewer different models of heat transfer units than has been possible with prior art heat transfer units. The inventor envisages that a single model of heat transfer unit will be suitable for the feet of at least 99% of all adults.

In some embodiments, the heat transfer unit has one or two fasteners at a periphery of the front left or right portion, one fastener at the front end of the first portion and one fastener at the rear end of the rear left or right portion. In some embodiments, only the fastener at the rear end of the rear left or right portion is (at least partly) stretchable whilst the remaining fasteners are substantially not stretchable.

Fasteners other than hook and loop fasteners may be used, for example fasteners that have an adhesive portion for (temporarily) adhering to another surface. Additionally or alternatively, the heat transfer unit can be secured to the foot in other ways, for example using elastic bands, adjustable straps and the like. These may either be attached to the heat transfer unit or be entirely separate items.

In one embodiment, the heat transfer unit comprises compartmentalization lines, in particular welding lines or fusion lines, for compartmentalizing the fluid chamber, in particular in order to guide the heat transfer fluid along a desired path through the fluid chamber.

At (or along) the compartmentalization lines, the first and second layers of material are connected to one another, for example using the same or a similar technique as the technique used for connecting the first and second material layers at the connection locations. In particular, the joining of the first and second material layers at the connection locations, along the periphery of the heat transfer unit and along the compartmentalization lines may be performed simultaneously, in particular using the same processing apparatus.

Whilst it is preferred that, at the compartmentalization lines, the first and second layers of material are connected in a fluid tight manner, it would also be acceptable for there not to be a 100% fluid tight seal along the compartmentalization lines. This would simply mean that (a relatively small proportion of) the heat transfer fluid might “take a shortcut” instead of following the desired path. However, the fluid flow across any compartmentalization lines should be significantly smaller than the fluid flow along the desired path, for example no more than 20%, 10% or 5% of the total fluid flow.

In one embodiment, the desired path has several curves or turns, in particular wherein the desired path is a labyrinthine path.

It is envisaged that the width of each individual path section is significantly smaller than the width of the entire heat transfer unit. This may help to ensure that the heat transfer fluid flows along substantially the entire (internal) surface of the transfer unit. By way of contrast, if each individual path section had a relatively large width, the likelihood of the fluid flow not reaching certain areas would increase, for example near the periphery of such a wide path section.

In one embodiment, the heat transfer unit comprises one or more additional layers, in particular an (additional) inner layer and/or an (additional) outer layer, in particular wherein the inner layer is arranged to face towards the foot and/or the outer layer is arranged to face away from the foot, in particular wherein the inner layer and/or the outer layer comprises a fabric layer.

Providing an additional inner layer may make the heat transfer unit more comfortable to wear, in particular if the first and second material layers are made from a plastics material.

An additional outer layer, again for example made from fabric, may help to ensure that one portion of the heat transfer unit can (temporarily) be fastened to another portion, in particular if a hook and loop fastener is used. To this end, one portion of the heat transfer unit may be provided with the “hook” element of a hook and loop fastener, and another portion of the heat transfer unit may be provided with the “loop” element of a hook and loop fastener.

In one embodiment, the heat transfer unit is suitable for feet of EU shoe size 28 to 46 or 47 and/or for feet with a length (of the sole or overall) from 17 or 18 cm to 30 cm, in particular wherein the heat transfer unit can cover at least 80% of the entire surface of the foot below the ankle, in particular at least 85%, 90% or 95%.

Being able to cover as much of the surface of the foot as possible may help to ensure sufficient and effective transfer of heat to/from the foot. Further, the adjustability of the heat transfer unit over a relatively wide range of feet sizes/shapes may mean that a larger range of feet sizes/shapes can be covered with a single model of the heat transfer unit than has been possible with some prior art devices.

Ideally, the ability of the heat transfer unit to cover a large percentage of the entire surface of the foot below the ankle will also result in the heat transfer unit, in particular the first material layer or any additional inner layer, being able to be in direct contact with the foot. The surface in direct contact is expected to be (slightly) smaller given that small pockets of air may remain between the foot and the heat transfer unit, for example in or near a region of overlap between different portions of the heat transfer unit, or where the heat transfer unit develops a crease as it is wrapped around the foot. It is envisaged that the heat transfer unit can be in direct contact with at least 70% of the entire surface of the foot below the ankle, in particular at least 75%, 80%, 85%, 90% or 95%.

In one embodiment, the heat transfer unit comprises an input port and an output port for connection to a heating or cooling device, to allow the heat transfer fluid to flow between the heating or cooling device and the heat transfer unit.

The input and output ports can be permanently connected to conduits such as hoses or similar for connection to a heating or cooling device. Alternatively, the input and output ports are suitable for temporary connection to such a conduit. Suitable input/output ports are known in the art and will therefore not be described in detail.

In one embodiment, the fluid chamber has a capacity of less than 400 ml, in particular less than 350 ml or 325 ml, in particular wherein the fluid chamber has a capacity of about 300 ml.

The inventor has found that this volume is sufficient to ensure good contact between the heat transfer unit and an average human foot. As mentioned above, a reduction of the volume of the fluid chamber may mean that the capacity of any heating or cooling device to be used with the heat transfer unit can also be reduced accordingly and/or that replenishing of heat transfer fluid during operation becomes unnecessary. Further, the reduced volume of the fluid chamber of the heat transfer unit may also enable two heat transfer units to be operated with one (i.e. the same) heating or cooling device at the same time, with the heat transfer units connected in parallel or in series. It may also be possible to operate more than two heat transfer units with one heating or cooling device at the same time, for example two heat transfer units of the type described herein (respectively for two feet), as well as one or two additional heat transfer units respectively for one or two hands.

In one embodiment, when the heat transfer unit is used in conjunction with a heat transfer fluid having a temperature of 15° C. in order to cool an average adult foot, a surface temperature of the foot reaches 22° C. or lower within 15 minutes of operation of the heat transfer unit.

This assumes that the heat transfer unit is used in an environment having normal room temperature.

The properties of the heat transfer unit, in particular the maximum possible flow rate of the heat transfer fluid through the heat transfer unit and/or the rate of heat transfer between the fluid chamber and a surface of the heat transfer unit facing towards the foot, may be such that when the heat transfer unit is used in conjunction with a heat transfer fluid having a temperature of 15° C. in order to cool an average adult foot, a surface temperature of the foot reaches 22° C. or lower within 15 minutes of operation of the heat transfer unit.

If the heat transfer unit is used to cool a human foot, it is possible to reduce or even prevent at least some of the negative effects of chemotherapy, in particular chemotherapy-induced peripheral neuropathy (CIPN) and hand-foot syndrome (HFS).

According to a second aspect, the present invention provides a heat transfer apparatus comprising:
a heat transfer unit according to the first aspect or any embodiments or variants thereof;
a heating or cooling device for heating or cooling a heat transfer fluid; and
at least one conduit, in particular two conduits, for connecting the heat transfer unit to the heating or cooling device to allow the heat transfer fluid to flow between the heating or cooling device and the heat transfer unit.

According to a third aspect, the present invention provides a method of heating or cooling a human foot, wherein the method comprises:
providing a heat transfer unit according to the first aspect or any embodiments or variants thereof;
placing a human foot on the first region;
bending the heat transfer unit so as to wrap the heat transfer unit around the foot; and
causing a heat transfer fluid to flow into the heat transfer unit, to flow through the fluid chamber and to flow out of the heat transfer unit.

The above steps can be carried out in the stated order or a different order. For example, it is possible to start the fluid flow (i.e. causing the heat transfer fluid to flow into the heat transfer unit, through the fluid chamber and out of the heat transfer unit) before wrapping the heat transfer unit around the foot (or even before placing the foot on the first region).

The method of heating or cooling a human foot can either be used in the context of a therapeutic treatment, or alternatively for non-therapeutic purposes.

Further advantages, features and applications of the present invention are described in the following detailed description and the appended figures, wherein:

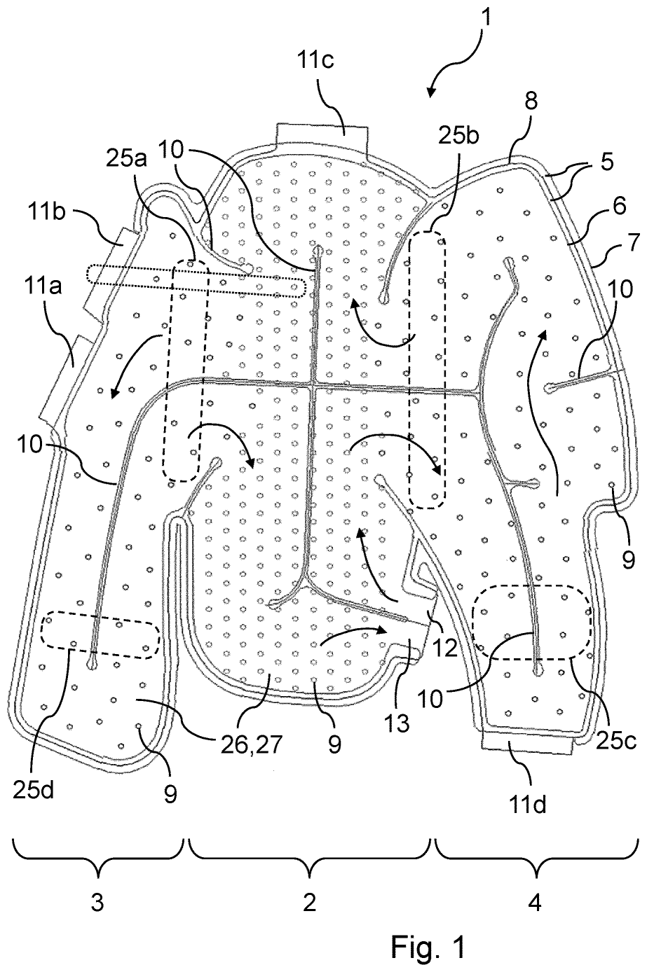

FIG. 1 schematically shows a top view of a heat transfer unit in an unfolded condition in accordance with an embodiment of the present invention.

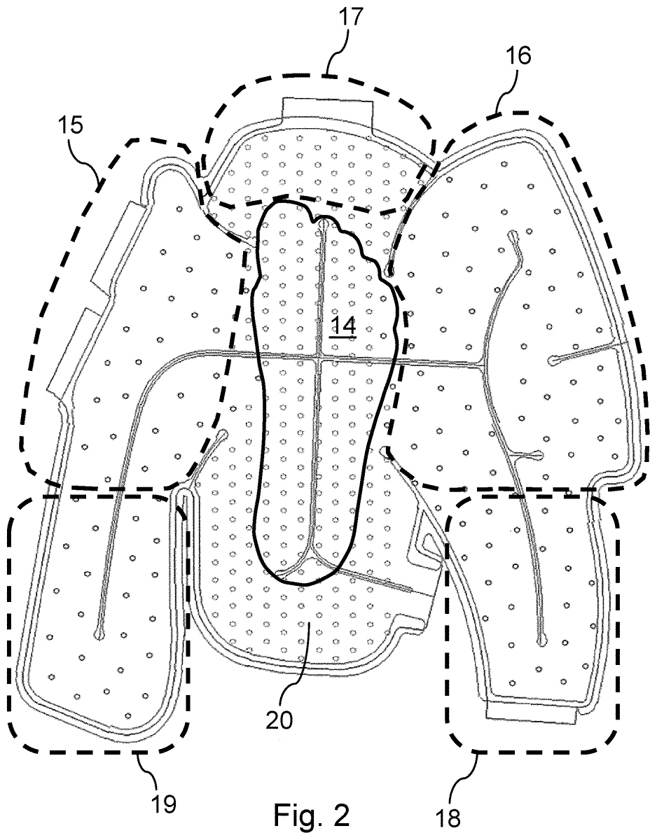

FIG. 2 schematically shows a further top view of the heat transfer unit of FIG. 1 in an unfolded condition.

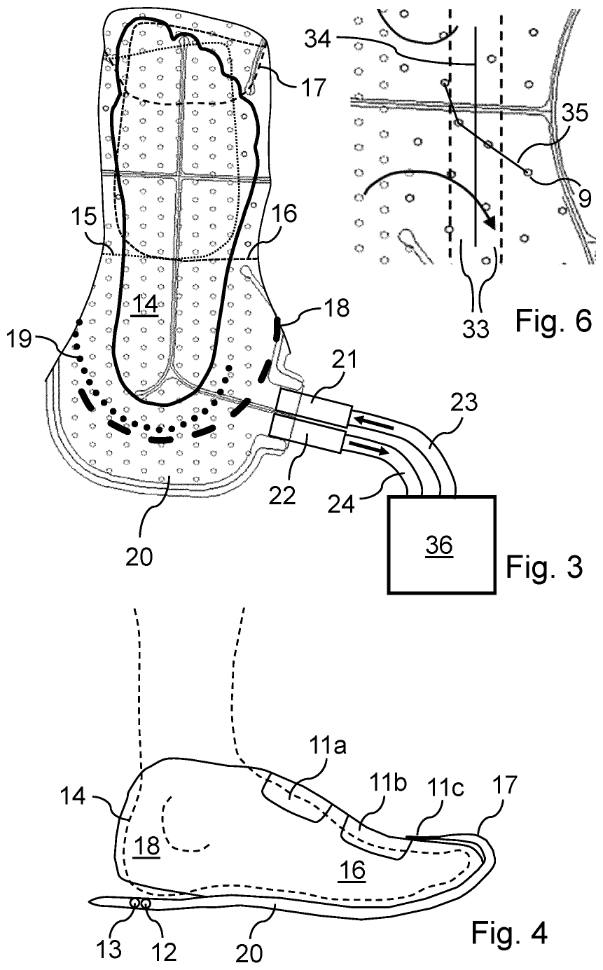

FIG. 3 schematically shows a heat transfer apparatus including a top view of the heat transfer unit of FIG. 1 in a folded condition.

FIG. 4 schematically shows a side view of the heat transfer unit of FIG. 1 when applied to a foot, as seen from the right side of the foot.

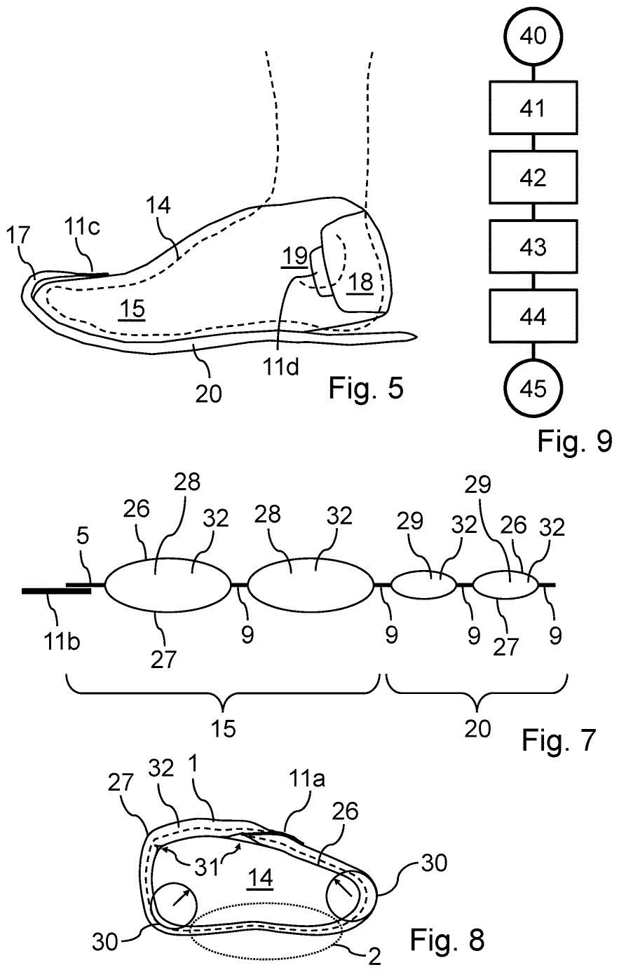

FIG. 5 schematically shows a side view of the heat transfer unit of FIG. 1 when applied to a foot, as seen from the left side of the foot.

FIG. 6 schematically shows an enlarged top view of a portion of the heat transfer unit of FIG. 1 in an unfolded condition.

FIG. 7 schematically shows an enlarged cross-sectional view of a portion of a heat transfer unit in accordance with an embodiment of the present invention.

FIG. 8 schematically shows a cross-sectional view of a heat transfer unit in accordance with an embodiment of the present invention, the heat transfer unit being applied to a foot.

FIG. 9 shows a flowchart illustrating a method in accordance with an embodiment of the present invention.

In the figures, identical reference signs are used for the same or similar elements of the embodiments of the invention described herein. To the extent that an element is shown in more than one figure, this element and its function will not necessarily be explained repeatedly.

FIG. 1 schematically shows a top view of a heat transfer unit 1 in an unfolded, substantially flat condition in accordance with an embodiment of the present invention. The heat transfer unit 1 comprises first and second material layers 26, 27, one on top of the other. Both material layers 26, 27 are made from a flexible, substantially fluid tight material, for example LDPE. Since FIG. 1 is a top view, the first and second material layers 26, 27 are not shown separately in this figure.

The first and second material layers 26, 27 are separately shown in FIG. 7, which schematically shows an enlarged cross-sectional view of a portion of the heat transfer unit 1. Reference should be made to both figures.

The first and second material layers 26, 27 are connected to one another at their periphery 5, in various connection locations 9 and along compartmentalization lines 10, each of which will be described below. In order to connect the first and second material layers 26, 27 in these locations, the material layers 26, 27 can for example be fused or welded together or adhered to one another using an adhesive. In one example, the two material layers 26, 27 are fused together through the application of pressure and radiation (such as heat), whereby the connections along the periphery 5, at the connection locations 9 and along the compartmentalization lines 10 can be made simultaneously using the same processing apparatus. In locations other than the periphery 5, the connection locations 9 and the compartmentalization lines 10, the first and second material layers 26, 27 remain separate and simply lie one on top of the other, forming passageways 28, 29 to enable a heat transfer fluid to flow therethrough. As will be described in more detail later, the passageways 28, 29 have different sizes, in particular depending on the distance between the connection locations 9, the fused periphery 5 and the compartmentalization lines 10. In the example shown in FIG. 7, the heat transfer unit 1 has large spaces or large passageways 28 and small spaces or small passageways 29. All passageways together effectively form the fluid chamber 32 bounded by the first and second material layers 26, 27. Whilst the cross-sectional view of FIG. 7 shows the passageways 28, 29 as individual elements, it will be apparent from FIG. 1 that the passageways are (all) connected, given the matrix-like configuration of the individual connection locations 9.

FIG. 1 shows three lines along most of the periphery 5. In the embodiment shown, the first and second material layers 26, 27 are connected across the entire space between the inner line (inner edge) 6 and the outer line (outer edge) 7 of the periphery 5. To this end, the first and second material layers 26, 27 can be placed one on top of the other and then connected as described above, whereby the first and second material layers 26, 27 may either have the size and shape as indicated by the outer edge 7, or may be larger. The first and second material layers 26, 27 are then cut along the middle line 8. The material of the first and second material layers 26, 27 that has been cut off (i.e. between the middle line 8 and the outer line 7) can then be discarded. Despite the material between the middle line 8 and the outer line 7 normally being discarded (i.e. this discarded material would not be part of the finished product), the outer line 7 is shown in several of the drawings.

As shown in FIG. 1, the fused periphery 5 is interrupted in one location towards the rear of the heat transfer unit 1. This interruption serves as input and output ports 12, 13. Conduits (not shown in FIG. 1) can be connected to the input port 12 and the output port 13 so that heat transfer fluid can be supplied into, and removed from, the heat transfer unit 1. Several arrows generally indicate the direction of flow of the heat transfer fluid (or a desired fluid path) through the fluid chamber 32 of the heat transfer unit 1.

The fluid path has several turns or curves, resulting in a serpentine pattern or labyrinthine path. The fluid path is primarily defined by the inner edge 6 of the periphery 5 and a number of compartmentalization lines 10. Some of the compartmentalization lines 10 are joined onto the periphery 5 of the heat transfer unit 1, whilst some others are not, such as the generally cross-shaped structure of compartmentalization lines 10 towards the middle of the figure, together with its “branches”.

The compartmentalization lines 10 help to ensure that the heat transfer fluid flows across substantially the entire surface of the heat transfer unit 1. The width of each section of the fluid path formed by the compartmentalization lines 10 and the periphery 5 is significantly smaller than the overall width of the heat transfer unit 1.

Whilst the input and output ports 12, 13 are shown in a particular location towards the rear of the heat transfer unit 1, on the right side of a central portion 2 of the heat transfer unit 1, the input and output ports 12, 13 could also be provided at a different location, either at a different location along the periphery 5 or even within (only) one of the first and second material layers 26, 27 (i.e. not integrated into the periphery 5). Further, the arrangement of the compartmentalization lines 10 as shown in FIG. 1 is only one example. A different arrangement of compartmentalization lines 10, perhaps with more, or fewer compartmentalization lines 10, is also conceivable. Also, the direction of flow of the heat transfer fluid along the fluid path could be reversed by using port 13 as the input port and using port 12 as the output port.

As indicated in FIG. 1, the heat transfer unit 1 generally has a central portion 2, a left portion 3 and a right portion 4. The left portion 3 is joined onto the left side of central portion 2 along part of its length. Similarly, the right portion 4 is joined onto the right side of central portion 2 along part of its length. As will be explained in more detail later, when the heat transfer unit is to be applied to a human foot, the foot would be placed on central portion 2 and the left portion 3 and the right portion 4 would be folded or bent upwards so that they extend respectively along the left and right sides of the foot.

The central portion 2 and the left and right portions 3, 4 are provided with connection locations 9. The connection locations 9 across most of the central portion 2 form a substantially regular pattern with a first average spacing of the connection locations 9. The region in which the connection locations 9 have the first average spacing is herein referred to as a first region. The average distance between nearest neighbors in the first region may be about 1 cm. In variants of this embodiment, the first average distance may have different values, for example between 0.5 cm and 1.5 cm. Further, the pattern formed by the connection locations 9 in the first region does not necessarily have to be as regular as shown in FIG. 1.

The connection locations 9 in the left and right portions 3, 4 have a second average spacing, which is greater than the first average spacing of the connection locations 9 in the central portion 2. The region in which the connection locations 9 have the second average spacing is herein referred to as a second region, in the embodiment shown in FIG. 1 made up of two individual regions (one on the left, one on the right). For example, the second average distance between nearest neighbors of connection locations 9 in the second region can be about twice the first average distance. In variants of this embodiment, the second average distance between nearest neighbors of connection locations 9 in the second region may be between 1.5 to 2.5 times the first average distance, or even greater. The pattern formed by the connection locations 9 in the second region may be a regular pattern, or alternatively an irregular pattern.

The description will now also refer to FIG. 2, which schematically shows a further top view of the heat transfer unit 1 of FIG. 1 in an unfolded condition. In FIG. 2, several regions of the heat transfer 1 have been encircled by dashed lines. A base portion 20 is indicated at the center of the heat transfer unit 1. This largely corresponds to the central portion 2, except where other regions 15 to 19 are indicated in FIG. 2. In normal use, the base portion 20 would be placed on a flat surface and a human foot 14 would be placed thereupon. Joined onto the left side of the base portion 20 (and somewhat towards the front of the heat transfer unit 1) is a front left portion 15. In turn, a rear left portion 19 is joined onto the rear end of the front left portion 15 without direct connection between the rear left portion 19 and the base portion 20. In other words, a gap is formed between the rear left portion 19 and the base portion 20.

Similarly, joined onto the right side of the base portion 20 (and somewhat towards the front of the heat transfer unit 1) is a front right portion 16. In turn, a rear right portion 18 is joined onto the rear end of the front right portion 16 without direct connection between the rear right portion 18 and the base portion 20. Again, a gap is formed between the rear right portion 18 and the base portion 20.

At the front of the base portion 20 there is a front portion or toe portion 17.

Given the flexibility of the heat transfer unit 1, the front left portion 15, the front right portion 16 and the toe portion 17 can be bent or folded upwards over the front part of the foot 14. It is envisaged that, after a foot 14 has been placed on the base portion 20, generally as indicated in FIG. 2, the front right portion 16 is first folded over the front part of foot 14. The front left portion 15 is then also folded over the front part of foot 14, at least partly overlapping front right portion 16. In order to secure front left portion 15 in place, front left portion 15 is provided with two fasteners 11a and 11b (FIG. 1), which can be hook and loop fasteners. That is, one part of the hook and loop fastener, for example the hook part, may be (permanently) attached to the periphery of front left portion 15, and the other part of the hook and loop fastener, for example the loop part, may be (permanently) attached to what would be the underside of front right portion 16 in FIG. 2. In this way, when the front left portion 15 is folded over onto front right portion 16 and the hook part attached to the loop part, this not only secures front left portion 15 (temporarily) in place, but also front right portion 16.

A dedicated loop part does not need to be provided on front right portion 16 if the material of front right portion 16 itself is such that the hook part of the hook and loop fastener can directly be attached to that material. Further, other types of fasteners may be provided, including adhesive fasteners. It is also possible to secure the front left and right portions 15, 16 in place using a separate element such as an elastic band or adjustable strap or similar.

The toe portion 17 is then also folded over the front part of foot 14, i.e. over part of front left portion 15, and also secured in place in like manner, for example using fastener 11c (permanently) attached to the periphery of toe portion 17.

Once the front left and right portions 15, 16 have been folded upwards and partly over the front part of foot 14, the rear left and right portions 19, 20 will be in a generally upright orientation, approximately to the left and right of the rear part of foot 14, approximately left and right of the ankle or below. Rear left portion 19 is then bent around the back of foot 14, around or somewhat above the heel or around the Achilles tendon of foot 14. Similarly, rear right portion 18 is also bent around the back of foot 14, at least partly overlapping rear left portion 19. Rear right portion 18 is then also secured in place, for example using fastener 11d attached to the rear end of rear right portion 18. Fastener 11d may again be constructed and used in a similar way as fasteners 11a, b and c.

In a variant, one or more or all of the fasteners 11a-d are (partly) stretchable. For example, fastener 11d could be (partly) stretchable while the other fasteners such as fasteners 11a-c are substantially not stretchable. Fastener 11d can, for example, comprise a substantially non-stretchable portion at what is shown in FIG. 1 as the lower (i.e. “free”) end, as well as a stretchable portion between that lower/free end and the periphery 5 of the first and second flexible material layers 26, 27. In this context it is to be noted that FIG. 1 does not necessarily show the fasteners (such as fastener 11d) to scale. They/it may be significantly longer than indicated in FIG. 1, in particular if they/it comprise(s) a stretchable portion (e.g. elasticated fabric) and a substantially non-stretchable portion (e.g. non-elastic fabric).

FIGS. 3 to 5 respectively show schematic top, right side and left side views of the heat transfer unit 1 of FIG. 1 when applied to a foot 14, i.e. in a folded or bent condition.

In FIG. 3, the base portion 20 is shown in solid lines. The front right portion 16 is indicated by a dashed line and is shown as covering much of the front part of foot 14. The front left portion 15 is indicated by a dotted line and is also shown as covering much of the front part of foot 14. The front left portion 15 at least partially overlaps the front right portion 16, as described above. The toe portion 17 is also indicated by a dashed line and is shown as covering the toe region of foot 14, overlapping front right portion 16 and front left portion 15. The rear left portion 19 is indicated by a dotted line extending partway around the back of foot 14. Since the rear left portion 19, when applied to foot 14, is in a substantially upright orientation, only the profile of rear left portion 19 is indicated in FIG. 3. Similarly, the rear right portion 18 is indicated by a dashed line, also extending partway around the back of foot 14 and overlapping rear left portion 19. Rear right portion 18 is also in a substantially upright position, and therefore only its profile is indicated in FIG. 3.

FIGS. 4 and 5 further illustrate the heat transfer unit 1 when applied to, or wrapped around, foot 14. These figures also generally show the position of fasteners 11a-d securing the various portions of the heat transfer unit 1 in place.

It will be appreciated that variants of this embodiment may have a different arrangement of fasteners. For example, one or more fasteners could be provided on front right portion 16, either in addition to, or instead of, the fasteners 11a, 11b on front left portion 15. Similarly, one or more fasteners could be provided on rear left portion 19, either in addition to, or instead of, the fastener 11d on rear right portion 18. Likewise, the order in which the various portions of heat transfer unit 1 are folded over, or around, foot 14 may be different.

Considering all of FIGS. 1 to 5 together, it can be seen that the left portion 3 (FIG. 1) or the front left portion 15 (FIGS. 2, 3) is folded or bent in particular in a region 25a indicated in FIG. 1. Similarly, the right portion 4 (FIG. 1) or the front right portion 16 (FIGS. 2, 3) is folded or bent in particular in a region 25b indicated in FIG. 1. As will be explained below, the radius of curvature may be smallest in these regions 25a, b. They are therefore regions at risk of kinking- or they would normally be at risk of kinking. However, to reduce the risk of kinking and therefore to reduce the risk of the flow of heat transfer fluid across these regions being impeded or even interrupted, the regions 25a, b, where the smallest radius of curvature is likely to be found, is in the second region, i.e. the region in which the average spacing of the connection locations 9 is greater than in the first region (central portion 2).

In a similar vein, a relatively sharp bend will be present in the rear left and right portions, 19, 18, where they wrap around the Achilles tendon. This is indicated in FIG. 1 by regions 25c, d. Again, the average spacing of the connection locations 9 in the rear left and right portions 19, 18 (and in particular in the regions at risk of kinking 25c, d) is greater than in the first region.

The exact location of relatively sharp bends will of course depend on the size and shape of the foot to which the heat transfer unit 1 is to be applied. Therefore, the second region, in which the average spacing of the connection locations 9 is greater than in the first region, is made sufficiently large so as to accommodate a relatively wide range of feet sizes/shapes whilst reducing the risk of kinking.

FIG. 3 also shows an input hose or conduit 23 and an output hose or conduit 24 for supplying heat transfer fluids to/from the heat transfer unit 1. The conduits are respectively provided with an input connector 21 and an output connector 22 for respective connection to input port 12 and output port 13 of heat transfer unit 1. At the opposite end, conduits 23, 24 are connected to a heating or cooling device 36, which is provided to heat or cool the heat transfer fluid and to circulate it through the heat transfer unit 1. The heating or cooling device 36 is not shown to scale-it would typically be significantly bigger than heat transfer unit 1.

Referring again to FIG. 7, it can now be appreciated from the foregoing description that what, by way of example, is shown in FIG. 7 can be regarded as a cross-sectional view of a small portion of the heat transfer unit 1 near the top left corner of FIGS. 1 and 2. The relevant portion is encircled in FIG. 1 using a dotted line (not labelled). The left-hand side of this dotted line shape corresponds to the left-hand end of what is shown in FIG. 7, and the right-hand side of the dotted line shape corresponds to the right-hand end of FIG. 7. As mentioned above, FIG. 7 shows a series of connection locations 9, whereby the distance between the connection locations 9 is smaller in the central portion 20 than in the front left portion 15. As a result, the passageways 29 between the connection locations 9 in the central portion 20 are also smaller than the passageways 28 between the connection locations 9 in the front left portion and between the periphery 5 and a connection location 9.

The fastener 11b can be seen in FIG. 7 as being attached to periphery 5. Fastener 11b (and any of the other fasteners described herein) may, for example, be sewn onto the periphery 5 or attached thereto in any other suitable way.

FIG. 8 schematically shows a cross-sectional view of a heat transfer unit 1 in accordance with an embodiment of the present invention, the heat transfer unit 1 having been applied to a foot 14. FIG. 8 may, for example, show a cross-sectional view of the foot and folded heat transfer unit 1 in a plane perpendicular to the drawing plane of FIG. 2 and approximately extending along the (in FIG. 2) horizontal part of the generally cross-shaped structure of compartmentalization lines 10 in the middle of the figure. The compartmentalization lines 10 and connection locations 9 are however not shown in FIG. 8. Instead, FIG. 8 shows the heat transfer unit 1 with its first or inner material layer 26 contacting the foot 14 and the second or outer material layer 27 facing away from the foot. The fluid chamber 32 is formed between the first and second material layers 26, 27. The approximate middle between the first and second material layers 26, 27 is indicated by a dashed line (not labelled).

As described above, the heat transfer unit 1 comprises a central portion 2 (approximate location indicated by a dotted line ellipse in FIG. 2), on which foot 14 is placed. Even when the heat transfer unit 1 is in use/operation, the central portion 2 of the heat transfer unit 1 is relatively flat, in particular in comparison to some other regions of the heat transfer unit 1. To the left and right of the central portion 2, the heat transfer unit 1 extends generally upwards and around the foot 14, whereby an overlap at the dorsum of foot 14 results-this is where the front left portion 15 overlaps the front right portion 16. The front left portion 15 is held in place by fastener 11a.

Whilst embodiments of the present invention enable a good contact between transfer unit 1 and a foot 14, small air pockets 31 may develop between the first, inner material layer 26 and the surface of the foot 14, for example in the region of the overlap at the dorsum of the foot or at relatively sharp bends of the heat transfer unit 1. This is why the surface area or percentage of the surface of the foot 14 in direct contact with the heat transfer unit 1 is expected to be slightly smaller than the surface area or percentage of the surface of the foot 14 surrounded by the heat transfer unit 1.

FIG. 8 also indicates two bending lines 30. The bending lines 30 extend into the drawing plane, i.e. they run generally parallel to a longitudinal direction of foot 14. The bending lines 30 are lines along which the heat transfer unit 1, when applied to a foot 14, has a relatively sharp bend, i.e. the radius of curvature at the bending lines 30 is smaller than in other regions of the heat transfer unit 1. The radii of curvature at the two bending lines 30 are visualized by two circles, the arrow within each circle indicating the respective radius of curvature. The circle on the right is fitted to the second material layer 27, i.e. to the surface of the heat transfer unit 1 facing away from the foot 14. The circle on the left is fitted to the middle between the first and second material layers 26, 27. Other ways of determining the radius of curvature in various positions are also conceivable, for example at positions between the dashed mid-line and the second material layer 27.

Since the bending lines 30 are located within the front left portion 15 and the front right portion 16, where the spacing of the connection locations 9 is greater than in the central portion 2, the radii of curvature are greater than would be the case if the spacing of the connection locations 9 in the region of the bending lines 30 was the same as in the central portion 2. The greater minimum radii of curvature in turn reduce the risk of the fluid chamber 32 collapsing, i.e. the risk of the first and second material layers touching each other, thereby restricting the fluid flow through the fluid chamber 32.

For some purposes (one of which will be explained below), it may be useful to determine the location/orientation of a bending region or the axis of a bending region. One suggested approach for determining a bending region and its associated axis is as follows. First, points of the heat transfer unit 1 are determined at which the radius of curvature is smallest. This is done for several “slices” along the longitudinal axis of the foot, one such slice being shown in FIG. 8. For each of these points, a region that extends a selected distance either side (along the surface of the foot) of these points, e.g. 1 cm, can be regarded as an individual bending region (for that slice). Considering all such slices together, an (overall) bending region results, or typically two (overall) bending regions, one on each side of the foot 14. A straight line can then be fitted through each of these bending regions. Each of these straight lines would then represent the axis of the respective bending region.

FIG. 6, which schematically shows an enlarged top view of a portion of the heat transfer unit 1 of FIG. 1 in an unfolded condition, illustrates the location/orientation of a bending region 33 and the associated axis 34, and how knowledge of this location/orientation may be useful. In this context it is to be noted that (the location/orientation of) a bending line 30, bending region 33 and its associated axis 34 is not fixed for a given heat transfer unit 1. Instead, this depends on the size and shape of a foot 14 to which the heat transfer unit 1 is applied since, in normal use, the heat transfer unit 1 would generally follow the contour of the foot 14. The location/orientation of a bending line 30, bending region 33 and associated axis 34 may also depend on how a user, such as a healthcare worker, applies the heat transfer unit 1 to the foot 14. However, for the purpose of the present application, it is assumed that a foot 14 of average size and shape, in particular of an adult human being, is used as a reference and that the heat transfer unit 1 is wrapped substantially tightly around the foot 14 in such a way that a good/direct contact between the heat transfer unit 1 and as much of the surface of the foot 14 as possible, at least below the ankle, is achieved.

We now assume that a foot 14 has been placed on base portion 20 and that the heat transfer unit 1, in particular the front left and right portions 15, 16, has/have been folded over the dorsum of the foot 14 and secured in place (e.g. see FIG. 3). In view of FIG. 8, the smallest radius of curvature may be in the front right portion 16, near the interface between the front right portion 16 and the base portion 20. At least part of this region is shown in FIG. 6—although FIG. 6 shows the heat transfer unit 1 in the unfolded condition for a clearer illustration. A bending region 33 can then be determined based on the location of the smallest radius of curvature. This bending region 33 corresponds to the region between the two dashed lines in FIG. 6. Since the heat transfer unit 1 is bent or folded over, the bending region 33 substantially defines (part of) a generally cylindrical shape running approximately parallel to the side of the foot 14. The right-hand circle in FIG. 8 indicates a cross-section of this cylindrical shape. A straight line can now be fitted through this cylindrical shape, and this straight line then represents the axis 34 of the respective bending region 33. This axis 34 is indicated as a vertical straight line 34 in FIG. 6 in the middle between the two dashed lines. Of course, since FIG. 6 shows the heat transfer unit 1 in the unfolded condition, the axis 34 of the bending region 33 will be in a different location than what is shown in FIG. 6, but the orientation of the axis 34 of the bending region 33 would correspond to the orientation of the vertical straight line 34 in FIG. 6, i.e. the actual axis of the bending region 33 would run parallel to the vertical straight line 34 in FIG. 6.

The orientation of the bending line 30 or of the axis 34 of the bending region 33 can then also be compared with the orientation of a line 35 formed by nearest neighbors of connection locations 9. FIG. 6 shows one such line 35. Similar to a bending line 30, the line 35 of nearest neighbors is not visible as such on the heat transfer unit 1. Instead, it is an imaginary line. In order to determine the course of such a line 35, at least three connection locations 9 should be considered which represent nearest neighbors to one another. Using the example shown in FIG. 6, we start, by way of example, with the connection location 9 at the bottom right of the line 35. The nearest neighbor is the connection location 9 in a direction of approximately 10 o'clock (12 o'clock being towards the top of the figure). From this second connection location 9, the nearest neighbor lies again in a direction of approximately 10 o'clock. These three connection locations 9 would be sufficient to define a line 35 of nearest neighbors. A fourth connection location 9 could also be taken into account, as shown in FIG. 6. This lies in a direction of approximately 11 o'clock with respect to the third connection location 9. Further points could be added. In the example, the substantially straight line formed by the first three connection locations 9 could be considered to define the line 35 of nearest neighbors. Alternatively, a line fitted through all four connection locations 9 could be considered to define the line 35 of nearest neighbors.

As can be appreciated from FIG. 6, line 35 of nearest neighbors has a significantly different orientation compared with the axis 34 of the bending region 33. Line 35 and axis 34 include an angle of about 50°. This further helps to prevent the heat transfer unit 1 from collapsing in the bending region, for the following reason: A heat transfer unit 1 of the type described herein tends to bend (or collapse) more easily along a line of several connection locations 9, in particular a straight line of several connection locations 9. The pattern of connection locations 9 in the bending region 33 is chosen such that lines 35 of nearest neighbors of connection locations 9 are substantially misaligned with axis 34 so as not to facilitate collapsing of the heat transfer unit 1 along axis 34.

FIG. 9 shows a flowchart illustrating a method in accordance with an embodiment of the present invention. After the start 40 of the method, a heat transfer unit (such as heat transfer unit 1 described above) is provided in step 41. In step 42, a foot of a human 14 is placed on the first region (such as central portion 2 or base portion 20). In step 43, the heat transfer unit 1 is bent in order to wrap the heat transfer unit 1 around the foot 14. In step 44, a heat transfer fluid is then caused to flow into the heat transfer unit 1, to flow through the fluid chamber 32 and to flow out of the heat transfer unit 1. Thereafter, the method can end (step 45).

While at least one example embodiment of the present invention has been described above, it has to be noted that a great number of variations thereto exist. Furthermore, it is to be appreciated that the described example embodiments only illustrate non-limiting examples of how the present invention can be implemented and that it is not intended to limit the scope, the application or the configuration of the apparatuses and methods described herein. Rather, the preceding description will provide the person skilled in the art with instructions for implementing at least one example embodiment of the invention, whereby it has to be understood that various changes in the functionality and the arrangement of the elements of the example embodiment can be made without deviating from the subject-matter defined by the appended claims and their legal equivalents.

### LIST OF REFERENCE SIGNS

1 heat transfer unit
2 central portion
3 left portion
4 right portion
5 periphery/fused portion
6 inner edge of fused portion
7 outer edge of fused portion
8 cutting line/edge of finished product
9 connection locations, (welding/fusion) dots
10 compartmentalization lines, (welding/fusion) lines
11a-11d fasteners
12 input port
13 output port
14 foot
15 front left portion
16 front right portion
17 front portion/toe portion
18 rear right portion
19 rear left portion
20 base portion
21 input connector
22 output connector
23 input hose
24 output hose
25a-25d regions at risk of kinking
26 first material layer
27 second material layer
28 large space/passageway
29 small space/passageway
30 bending line
31 air pocket
32 fluid chamber
33 bending region
34 axis of bending region
35 line formed by nearest neighbors of connection locations
36 heating or cooling device
40-45 method steps

1. A heat transfer unit adapted to be applied to a human foot in order to transfer heat to/from the human foot,
wherein the heat transfer unit comprises a first flexible material layer and a second flexible material layer defining a fluid chamber therebetween, allowing a heat transfer fluid to flow through the fluid chamber,
wherein the first and second flexible material layers are connected to one another at a plurality of connection locations,
wherein, in a first region of the heat transfer unit, the connection locations have a first average spacing and, in a second region of the heat transfer unit, the connection locations have a second average spacing, wherein the first average spacing is smaller than the second average spacing,
wherein, in normal use of the heat transfer unit, the first region of the heat transfer unit is arranged to be located adjacent a sole of the human foot, and the second region of the heat transfer unit is arranged to be located adjacent a region of the human foot different from the sole of the human foot.

2. The heat transfer unit according to claim 1, wherein, in normal use, the heat transfer unit is arranged to be bent along one or more bending lines or bending regions resulting in the heat transfer unit having radii of curvature respectively associated with each bending line or bending region, wherein the one or more bending lines or bending regions having the smallest radius of curvature are located in the second region, in particular wherein the radius of curvature is measured at a surface of the heat transfer unit facing away from the human foot, or in the middle between the first and second flexible material layers, in particular wherein, in normal use:
the one or more bending lines or bending regions having the smallest radius of curvature are located along the front half of the left and/or right side of the human foot and/or along the Achilles tendon; and/or
the smallest radius of curvature is 2 cm or less, in particular 1.5 cm or less, in particular 1.3 cm or less.

3. The heat transfer unit according to claim 2, wherein, at the one or more bending lines or bending regions having the smallest radius of curvature, connection locations that are nearest neighbors do not, in normal use of the heat transfer unit, form a line which is aligned with the respective bending line or with a longitudinal axis of the respective bending region, in particular wherein a line formed by nearest neighbors of connection locations includes an angle of at least 5°, 10°, 15°, 20°, 25° or 30° with said respective bending line or said longitudinal axis.

4. The heat transfer unit according to claim 1, wherein the first region occupies an area that represents a fraction of the total area of the heat transfer unit, wherein said fraction is between an upper limit and a lower limit, wherein the upper limit is 55%, 50% or 45%, and wherein the lower limit is 25%, 30% or 35%, in particular wherein said fraction is about 40%.

5. The heat transfer unit according to claim 1, wherein the second region comprises one or more, in particular all of:
a rear left portion;
a front left portion;
a front right portion; and
a rear right portion.

6. The heat transfer unit according to claim 1, wherein the heat transfer unit comprises one or more fasteners, in particular three or four fasteners, in particular hook-and-loop fasteners, in particular at a periphery of the heat transfer unit, for temporarily attaching a portion of the heat transfer unit to another portion of the heat transfer unit.

7. The heat transfer unit according to claim 1, wherein the heat transfer unit comprises compartmentalization lines, in particular welding lines or fusion lines, for compartmentalizing the fluid chamber, in particular in order to guide the heat transfer fluid along a desired path through the fluid chamber.

8. The heat transfer unit according to claim 7, wherein the desired path has several curves or turns, in particular wherein the desired path is a labyrinthine path.

9. The heat transfer unit according to claim 1, wherein the heat transfer unit comprises one or more additional layers, in particular an inner layer and/or an outer layer, in particular wherein the inner layer is arranged to face towards the foot and/or the outer layer is arranged to face away from the foot, in particular wherein the inner layer and/or the outer layer comprises a fabric layer.

10. The heat transfer unit according to claim 1, wherein the heat transfer unit is suitable for feet of E11 shoe size 28 to 46 or 47 and/or for feet with a length (of the sole or overall) from 17 or 18 cm to 30 cm, in particular wherein the heat transfer unit can cover at least 80% of the entire surface of the foot below the ankle, in particular at least 85%, 90% or 95%.

11. The heat transfer unit according to claim 1, wherein the heat transfer unit comprises an input port and an output port for connection to a heating or cooling device to allow the heat transfer fluid to flow between the heating or cooling device and the heat transfer unit.

12. The heat transfer unit according to claim 1, wherein the fluid chamber has a capacity of less than 400 ml, in particular less than 350 ml or 325 ml, in particular wherein the fluid chamber has a capacity of about 300 ml.

13. The heat transfer unit according to claim 1, wherein, when the heat transfer unit is used in conjunction with a heat transfer fluid having a temperature of 15° C. in order to cool an average adult foot, a surface temperature of the foot reaches 22° C. or lower within 15 minutes of operation of the heat transfer unit.

14. A heat transfer apparatus comprising:
a heat transfer unit according to claim 1,
a heating or cooling device for heating or cooling a heat transfer fluid; and
at least one conduit, in particular two conduits, for connecting the heat transfer unit to the heating or cooling device to allow the heat transfer fluid to flow between the heating or cooling device and the heat transfer unit.

15. A method of heating or cooling a human foot, wherein the method comprises: providing a heat transfer unit according to claim 1;
placing the foot on the first region;
bending the heat transfer unit so as to wrap the heat transfer unit around the foot; and
causing a heat transfer fluid to flow into the heat transfer unit, to flow through the fluid chamber and to flow out of the heat transfer unit.
